(19) United States
(12) Patent Application Publication
(10) Pub. No.: US 2015/0289986 A1
(43) Pub. Date: Oct. 15, 2015

(54) FLANGED ENDPLATE FOR AN INTERVERTEBRAL DISC PROSTHESIS AND INTERVERTEBRAL DISC PROSTHESIS INCORPORATING SAME

(71) Applicant: K2M, INC., Leesburg, VA (US)

(72) Inventors: Casey K. Lee, Florham Park, NJ (US); George Makris, West Orange, NJ (US)

(21) Appl. No.: 14/646,605

(22) PCT Filed: Nov. 21, 2013
(86) PCT No.: PCT/US2013/071345

§ 371 Date: May 21, 2015

Related U.S. Application Data
(60) Provisional application No. 61/729,126, filed on Nov. 21, 2012.

(51) Int. Cl. A61F 2/44

(52) U.S. Cl. CPC A61F 2/442

(57) ABSTRACT

An endplate for an intervertebral disc prosthesis includes: a baseplate, sized and configured to fit within at least a portion of an intervertebral space, and an anterior portion having an outer vertebra-contacting surface extending outwardly from the baseplate at an acute angle with respect to an axis of the baseplate, and being provided with an opening configured to receive a fastener.

### BACKGROUND OF THE INVENTION

1. Field of the Invention

This invention relates to prostheses for replacing a human intervertebral disc, and more particularly to an endplate for such a prosthesis incorporating an anterior flange configured to cooperate with a resected surface on an adjacent vertebra in a human spinal motion segment.

2. Background Art

The human spinal column achieves its remarkable combination of strong support and appropriate flexibility by reason of its structure comprising bony vertebrae separated by intervertebral discs of softer and flexible tissue that allow limited motion between adjacent vertebrae in flexion-extension, lateral bending, and torsion. Each individual flexible element of the spine, comprising a pair of adjacent vertebrae separated by an intervertebral disc, constitutes a spinal motion segment. The proper function of such a spinal motion segment requires the intervertebral disc to provide proper separation between the vertebrae while allowing sufficient relative motion in the sagittal, coronal and transverse anatomical planes of the body. While each intervertebral disc typically performs its function effectively without conscious awareness, the disc and surrounding tissues are provided with ample innervation that informs the individual of any damage and/or malfunction by providing a pain signal.

The spinal regions most susceptible to painful pathology of the intervertebral disc are the cervical and lumbar regions. Such painful pathology is typically the result of some traumatic injury or age-related changes in the structure and function of the intervertebral disc.

The most common pathologic condition causing chronic low back pain and neck pain is degenerative disc disease (DDD), which is typically the result of age-related changes in the tissues constituting the intervertebral disc, with accompanying abnormalities, e.g., deformation, in the functional structures of the disc. Under such conditions, even normal movement between the adjacent vertebrae can cause pain, which may become chronic and sufficiently severe to result in significant disability. When non-invasive treatment fails to relieve chronic disabling back pain caused by such disease, recourse is had to surgical intervention. For some time, palliative surgical procedures such as disc excision, decompression, and/or spinal fusion have been performed to relieve intractable pain of patients with degenerative disc disease. More recently, artificial intervertebral disc prostheses have been developed, which have made it possible to replace a degenerated disc with such a prosthesis to achieve pain relief and restore anatomical function.

A number of factors must be considered in the design of an intervertebral disc prosthesis if a successful outcome of disc arthroplasty is to be expected. The prosthesis design must provide for proper positioning, correct alignment, congruent contact surface area, and immediate and long term post-operative prosthetic stability within the disc space. In particular, the conformation of the vertebra-contacting surface of the prosthesis at the vertebra-prosthesis interface is of significant importance, particularly for post-operative stability of the prosthesis in the intervertebral space. Experience has shown that the clinical results of intervertebral disc arthroplasty are closely correlated to the proper initial positioning of the disc prosthesis in the disc space and subsequent maintenance thereof. For instance, if an implanted disc prosthesis does not maintain a stable position within the intervertebral space, the patient may experience post-operative pain from the index operated level and accelerated disc degeneration in adjacent spinal motion segments, as well as formation of osteophytic growths on the vertebrae.

In order to secure firm fixation of a vertebral endplate to the adjacent vertebra in a spinal motion segment, the outer surface that contacts the adjacent vertebra, is often provided with projecting structures, e.g., spikes, fins, and the like, which engage the vertebral body.

In the anterior approach for implantation of an intervertebral disc prosthesis it is necessary to provide access to the intervertebral space in order to excise the defective disc and insert an intervertebral disc prosthesis. Such access can be provided, e.g., by distracting the vertebrae of a spinal motion segment to enlarge the intervertebral distance for convenient insertion of an intervertebral disc prosthesis. In certain cases, however, the anterior rim of cortical bone of one or both of the vertebrae in a spinal motion segment can obstruct access to the intervertebral space. This problem is particularly prevalent in intervertebral disc replacement procedures in the cervical spine, because the lower anterior rim of the superior vertebra often extends downward, thereby obstructing, at least partially, surgical access to the intervertebral space. Such obstruction tends to make intervertebral disc replacement in the cervical spine more difficult than similar procedures in other regions of the spine.

Accordingly, a need has continued to exist for an endplate design that can alleviate the problems experienced in implantation of known intervertebral disc prostheses.

### SUMMARY OF THE INVENTION

According to the invention, an endplate for an intervertebral disc prosthesis is provided that promotes a stable relationship between the prosthesis and the adjacent vertebrae after implantation.

In one of its aspects, the prosthesis endplate of the invention comprises:

a baseplate, sized and configured to fit within at least a portion of an intervertebral space, and

an anterior portion having an outer vertebra-contacting surface extending outwardly from the baseplate at an acute angle with respect to an axis of the baseplate, and being provided with an opening configured to receive a fastener.

Accordingly, one preferred feature of the invention is an endplate for an intervertebral disc prosthesis that includes anterior portion having an outer vertebra-contacting surface extending outwardly from the baseplate at an acute angle with respect to an axis of the baseplate, wherein the vertebra-contacting surface is designed and configured to contact a resected surface of an adjacent vertebra.

According to a further feature of the invention, an endplate for an intervertebral disc prosthesis can include an angled anterior flange designed and configured to contact a resected surface of an adjacent vertebra.

According to yet a further feature of the invention, an endplate for an intervertebral disc prosthesis is configured to facilitate reliable and easy positioning, alignment, preparation of a congruent contact surface, and better stabilization against axial, bending, torsion and translation particularly in the cervical spine.

Further features of the invention will be apparent from the description of the invention which follows.

### BRIEF DESCRIPTION OF THE DRAWINGS

FIG. 1 is an oblique view of an intervertebral prosthesis incorporating an embodiment of the endplate of the invention.

FIG. 2 is an anterior elevational view of the intervertebral prosthesis of FIG. 1.

FIG. 3 is a lateral elevational view of the interverteberal prosthesis of FIG. 1.

FIG. 4 is top plan view of the intervertebral prosthesis of FIG. 1.

FIG. 5 is a top plan view of another embodiment of the invention.

FIG. 6 is an anterior elevational view of the intervertebral prosthesis of FIG. 5.

FIG. 7 is a lateral elevational view of the intervertebral prosthesis of FIG. 5.

FIG. 8 is a schematic lateral view of a cervical spinal motion segment indicating a position for a resection of the anterior lower rim of the superior vertebra.

FIG. 9 is a schematic lateral view of a prosthesis of FIG. 5 implanted in a cervical spinal motion segment

### DETAILED DESCRIPTION OF THE INVENTION

An intervertebral prosthesis designed to replace a degenerated, damaged, or otherwise defective natural intervertebral disc, with retention of at least some of the function of the natural disc, typically incorporates a pair of endplates designed for firm fixation to the adjacent vertebrae of a human spinal motion segment, together with a core structure between the endplates which provides an appropriate separation between the endplates and allows for at least some relative motion therebetween.

According to the invention, an endplate for an intervertebral disc prosthesis capable of secure attachment to an adjacent vertebra has:

a baseplate, sized and configured to fit within at least a portion of an intervertebral space, and

an anterior portion having an outer vertebra-contacting surface extending outwardly from the baseplate at an acute angle with respect to an axis of the baseplate, and being provided with an opening configured to receive a fastener.

The anterior portion of the endplate can be provided with one or more openings for fasteners to provide secure attachment of the endplate to the adjacent vertebra.

According to a further embodiment of the invention, an endplate for an intervertebral disc prosthesis capable of secure attachment to an adjacent vertebra has:

a baseplate, generally sized and configured to fit within at least a portion of the posterior region of the intervertebral space, and

a flange extending anteriorly and outwardly from the anterior portion of the baseplate at an acute angle with respect to the axis of the baseplate, and being provided with an opening configured to receive a fastener.

In a further embodiment of the invention, the anterior flange may extend from an anterior edge of the baseplate.

In an embodiment of the invention, the anterior flange is sized and configured to mate with a complementary surface prepared on the anterior rim of the adjacent vertebra.

The anterior flange may also be provided with one or more openings for accepting a fastener, e.g., a bone screw or a staple, for securing the endplate to the adjacent vertebra.

The endplate of the invention may be employed as the superior or upper endplate in an intervertebral disc prosthesis, or as an inferior or lower endplate of such a prosthesis, or both. In an embodiment of the invention the flanged endplate of the invention may be employed as the superior or upper endplate in an intervertebral disc prosthesis, while the lower endplate of the prosthesis does not include an anterior flange.

In an intervertebral disc prosthesis employing the endplate of the invention, either the upper endplate or the lower endplate may be provided with projections for engaging a respective adjacent vertebra.

The inner side of the endplate of the invention may be connected to a core structure of an intervertebral prosthesis, which can position the endplates of the intervertebral disc prosthesis at a suitable spacing for replacement of the natural intervertebral disc. The core structure may also be designed to provide relative motion between the upper and lower endplates of the prosthesis, appropriate for retaining natural motion of the spinal motion segment as much as possible. In an embodiment of an intervertebral disc prosthesis employing the endplate of the invention, the core structure may be an elastomeric body, which may be attached to one or both of the endplates.

In an embodiment of the invention the outer side of the baseplate of the intervertebral prosthesis endplate may be provided with a generally dome-shaped surface to facilitate contact with the surface of an adjacent vertebra.

The intervertebral prosthesis endplate of the invention may be constructed from any material conventionally used for intervertebral prostheses, e.g., stainless steel, titanium, and the like. It may be manufactured by any conventional process for forming such structures, e.g., by machining, assembling from component parts by welding, or the like.

The invention also comprises a method for implanting an intervertebral prosthesis incorporating an endplate of the invention wherein the anterior rim of an adjacent vertebra is surgically prepared with a surface configured to mate with the angled flange of the endplate, and, thereafter, the prosthesis is implanted in a surgically prepared site in the intervertebral space. The intervertebral prosthesis may then be secured to an adjacent vertebra by a fastening device, e.g., a bone screw, extending through a hole in the flange and into the body of the adjacent vertebra.

The invention will now be illustrated by the accompanying drawings showing the use of the endplate of the invention in a prosthesis having an elastomeric core element. It will be understood that the illustrated embodiments are illustrative only, the scope of the invention being defined by the appended claims.

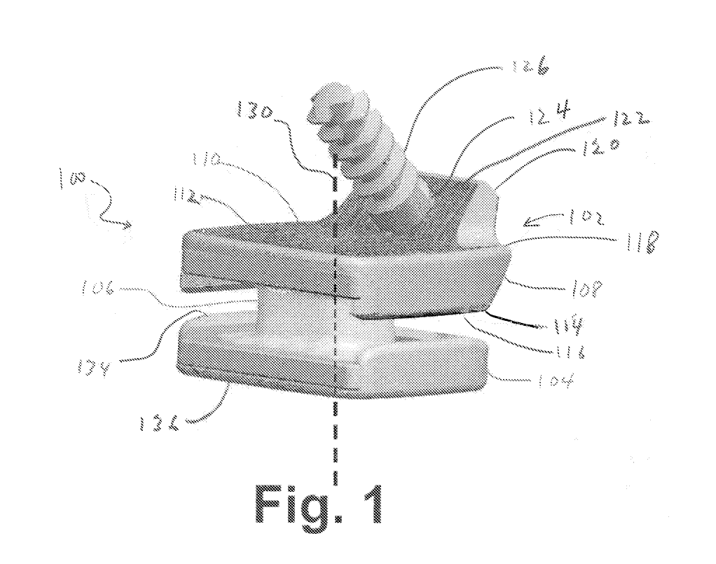

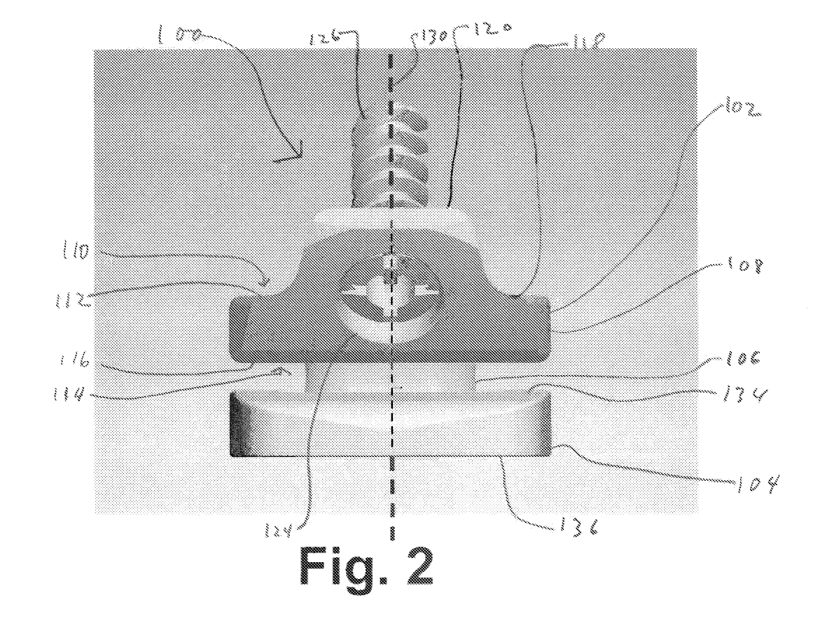

FIGS. 1-4 illustrate a prosthesis endplate of the invention and its use in an intervertebral prosthesis having an elastomeric core element.

The prosthesis 100 has an upper endplate 102, a lower endplate 104, and a core member 106, secured to the upper and lower endplates and which may be an elastomeric core member.

The endplate 102 includes a baseplate 108, which is sized and configured to be implanted in at least a posterior region of an intervertebral space. The baseplate 108 has an outer side 110 having an outer side surface 112 for contact with an adjacent superior vertebra, and an inner side 114 for securing the core member 106 to the endplate. The inner side 114 has an inner side surface 116. The core member 106 may include, for example, an elastomeric body, capable of supporting the axial loads placed on the spinal motion segment while permitting motion thereof in flexion, extension, rotation and lateral bending. Other materials and configurations of the core structure that maintain an appropriate separation of the prosthesis endplates are also suitable. See, for example, U.S. Published Patent Application No. 2008/0306609, the entire disclosure of which is incorporated herein by reference.

The baseplate 108 has an anterior portion 120 extending anteriorly and outwardly relative to the anterior edge 118 of the baseplate 108, preferably extending from the anterior edge as shown. In the form shown, anterior portion 120 is constituted by a flange. The flange 120 has an outer surface 122 designed and configured to contact a complementary surface formed on the adjacent vertebra. The flange 120 is fixed to the adjacent vertebra by appropriate surgical fasteners, e.g., a bone screw 126 inserted through an opening 124 in the flange 120. The outer surface 122 of the flange 120 is oriented outwardly at an acute angle 132 with respect to an axis 130 of the baseplate 108, as defined and discussed below. Any acute angle that provides enhanced secure orientation of the endplate to the adjacent vertebra is suitable. The angle 132 may be about 45 degrees and is preferably from about 30 to about 60 degrees.

The lower endplate 104 of the prosthesis 100 as shown has an inner side 134 for securing core member 106 and an outer side 136 for contact with an adjacent vertebra.

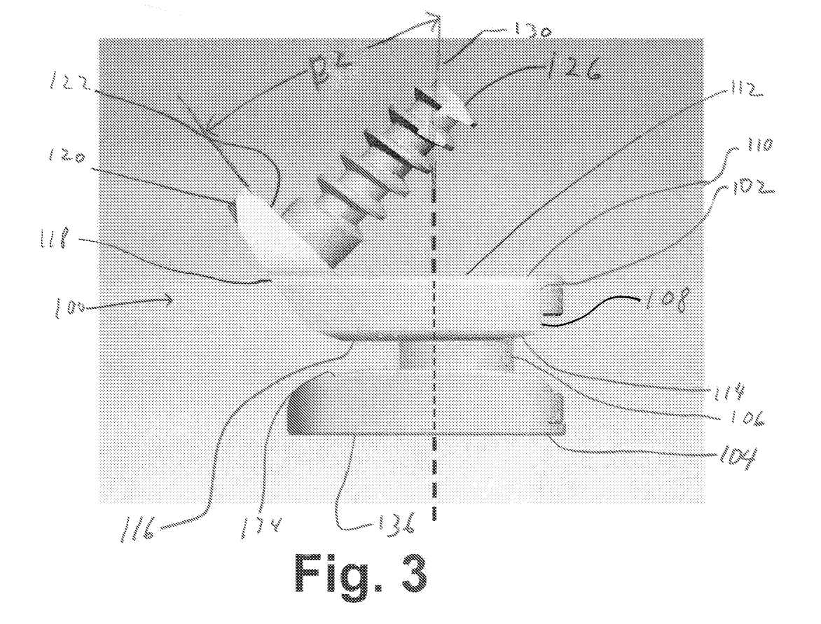

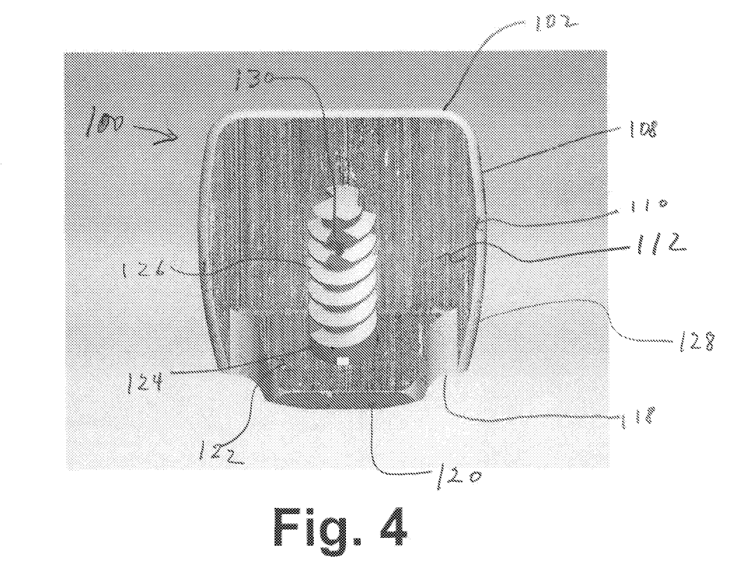

The baseplate 108 of the intervertebral prosthesis endplate 102 of the invention, when seen in plan view, as in FIG. 4, has a visible periphery 128. An axis 130 of the baseplate 108 is defined extending perpendicular to the plane of the baseplate 108 in plan view. The outer surface 122 of the anterior portion (flange) 120 is configured to form an acute angle 132 with the axis 130, as best seen in FIG. 3.

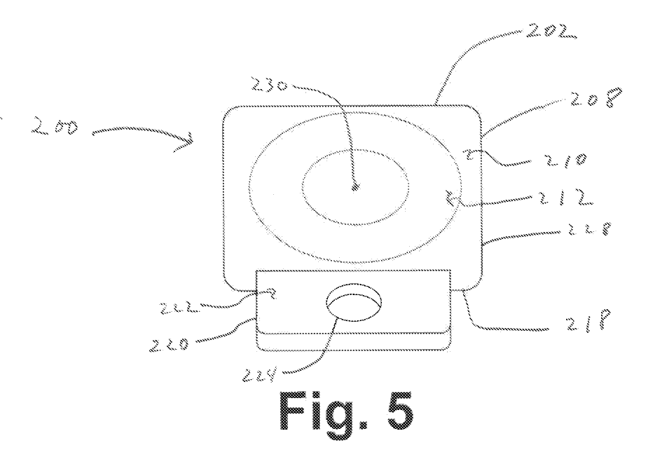

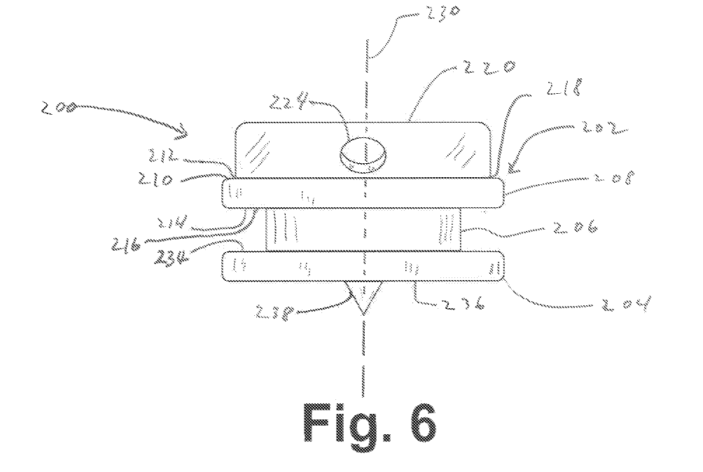

Another embodiment of the invention is illustrated in FIGS. 5-7. The illustrated intervertebral prosthesis 200 has an upper endplate 202, a lower endplate 204, and a core member 206, which may be a core structure including an elastomeric core body.

The endplate 202 includes a baseplate 208, which is sized and configured to be implanted in at least a posterior region of an intervertebral space. The baseplate 208 has an outer side 210 having an outer side surface 212 for contact with an adjacent superior vertebra, and an inner side 214 for attachment to the core member 206. The inner side 214 has an inner side surface 216. In the embodiment of FIGS. 5-7, the outer surface 212 of the outer side 210 of the base plate 208 has a domed shape. The core member 206 may include, for example, an elastomeric body, capable of supporting the axial loads placed on the spinal motion segment while permitting motion in flexion, extension, rotation and lateral bending. Other materials and configurations of the core structure that maintain an appropriate separation of the prosthesis endplates are also suitable. See, e.g., the aforementioned U.S. Published Patent Application No. 2008/0306609.

The baseplate 208 has an anterior portion (shown as a flange) 220 extending anteriorly and outwardly relative to the anterior edge 218 of the baseplate 208, preferably extending from the anterior edge as shown. The flange 220 has an outer surface 222 designed and configured to contact a complementary surface formed on the adjacent vertebra. The flange 220 is fixed to the adjacent vertebra by appropriate surgical fasteners, e.g., a bone screw 226 inserted through an aperture 224 in the flange 220. The outer surface of 222 of the flange 220 is oriented outwardly at an acute angle with respect to an axis 230 of the baseplate 208, as defined and discussed below. Any angle that provides enhanced secure orientation of the endplate to the adjacent vertebra is suitable. The angle 232 may be about 45 degrees.

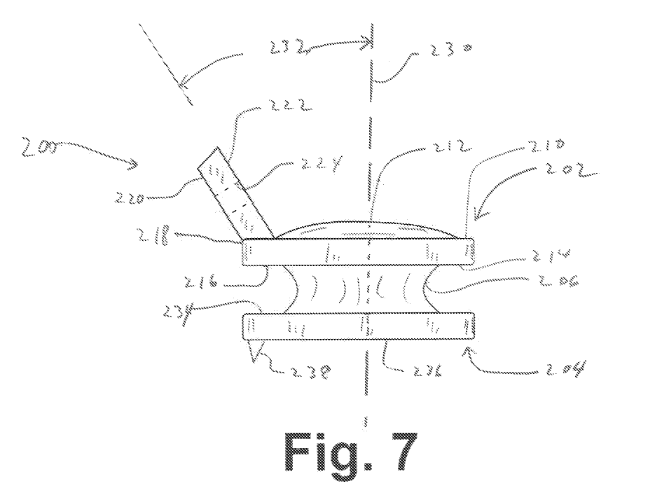

The baseplate of the intervertebral prosthesis endplate of the invention, when seen in plan view, as in FIG. 5, has a visible periphery 228. An axis 230 of the baseplate 208 is defined extending perpendicular to the plane of the baseplate 208 in plan view. The outer surface 222 of the anterior flange 220 is configured to form an acute angle 232 with the axis 230, as best seen in FIG. 7.

The lower endplate 204 of the prosthesis 200 as shown has an inner side 234 for attachment of core member 206 and an outer side 236 for contact with an adjacent vertebra. As indicated above, a lower endplate of a prosthesis incorporating an upper endplate of the invention may incorporate one or more projections, e.g., a spike 238, as shown, for engagement with an adjacent vertebra.

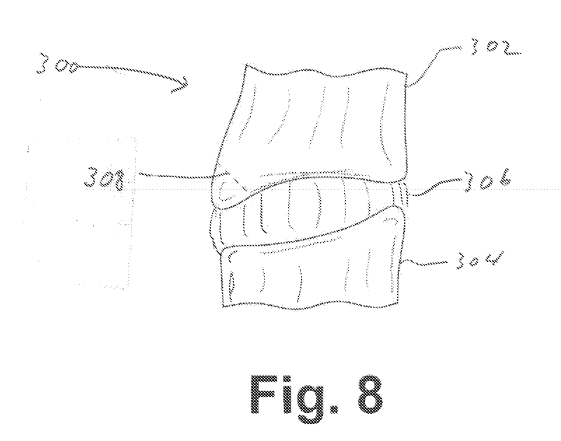

When an intervertebral prosthesis, e.g., the prosthesis 200, is to be implanted in an intervertebral space after removal of a diseased or degenerated natural intervertebral disc, an adjacent vertebra is prepared as indicated in FIG. 8. FIG. 8 schematically illustrates an anterior elevational view of a spinal motion segment 300, comprising a superior vertebra 302, an inferior vertebra 304, and a natural intervertebral disc 306. The dashed line indicates the location of a resected surface 308 to be formed on the lower anterior rim of the superior vertebra 302 that contacts the outer surface 222 of the anterior flange 220 when the intervertebral prosthesis endplate 202 is implanted.

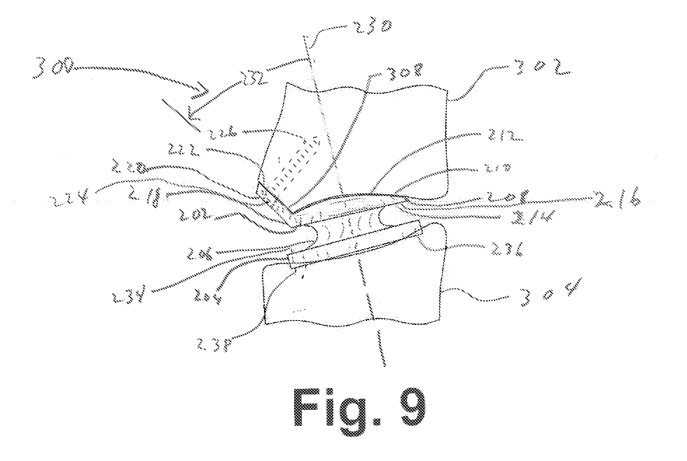

A schematic lateral elevational view of spinal motion segment 300 having an intervertebral disc prosthesis 200 implanted therein is shown in FIG. 9. The upper endplate 202 and lower endplate 204 of the intervertebral prosthesis 200 contact their respective adjacent vertebrae 302 and 304. The outer surface 222 of the anterior flange 220 contacts the resected surface 308 of the superior vertebra 302 and is fastened thereto by means of a bone screw 226.

The outer surface 222 of the anterior flange 220 and the complementary resected surface 308 are shown as generally planar surfaces. However, those skilled in the art will understand that other shapes for the complementary surfaces 222 and 308 are possible.

In addition to its various advantages as will be appreciated from the above discussion, the endplate of the invention provided with an angled anterior portion or flange as described allows for a low profile screw fixation between the endplate and the vertebral body by the use of an appropriately recessed screw. Furthermore, the outer surface of the anterior portion of the flange covers the resected surface of the superior vertebra, thus tending to prevent possible auto-fusion of a surgically exposed bone surface.

The invention having been described in terms of certain embodiments, it will be apparent to those skilled in this art that many changes and alterations can be made without departing from the spirit or essential characteristics of the invention. The present disclosure is therefore to be considered as illustrative, and not restrictive, of the invention.

We claim:

1. An endplate for an intervertebral disc prosthesis, comprising:
a baseplate, sized and configured to fit within at least a portion of an intervertebral space, and
an anterior portion having an outer vertebra-contacting surface extending outwardly from the baseplate at an acute angle with respect to an axis of the baseplate, and being provided with an opening configured to receive a fastener.

2. The endplate of claim 1, wherein said acute angle is from about 30 degrees to about 60 degrees.

3. The endplate of claim 1, wherein said acute angle is about 45 degrees.

4. The endplate of claim 1, wherein said anterior portion is a flange.

5. The endplate of claim 4, wherein said flange extends anteriorly relative to an anterior edge of said baseplate.

6. The endplate of claim 4, wherein said flange extends from an anterior edge of said baseplate.

7. The endplate of claim 1, wherein said baseplate is sized and configured to fit within at least a portion of a cervical intervertebral space.

8. An intervertebral disc prosthesis, comprising:
an upper endplate;
a lower endplate; and
a core, positioned between and secured to said upper endplate and said lower endplate,
wherein at least said upper endplate includes:
a baseplate, sized and configured to fit within at least a portion of an intervertebral space, and
an anterior portion having an outer vertebra-contacting surface extending outwardly from the baseplate at an acute angle with respect to an axis of the baseplate, and being provided with an opening configured to receive a fastener.

9. The intervertebral disc prosthesis of claim 8, wherein said acute angle is from about 30 degrees to about 60 degrees.

10. The intervertebral disc prosthesis of claim 8, wherein said acute angle is about 45 degrees.

11. The intervertebral disc prosthesis of claim 8, wherein said anterior portion is a flange.

12. The intervertebral disc prosthesis of claim 11, wherein said flange extends anteriorly relative to an anterior edge of said baseplate.

13. The intervertebral disc prosthesis of claim 11, wherein said flange extends from an anterior edge of said baseplate.

14. The intervertebral disc prosthesis of claim 8, wherein said prosthesis is sized and configured to fit within at least a portion of a cervical intervertebral space.

15. The intervertebral disc prosthesis of claim 8, wherein said lower endplate includes a projection for engagement with an adjacent vertebra.

16. The intervertebral disc prosthesis of claim 15, wherein said projection is a spike.

17. The intervertebral disc prosthesis of claim 8, wherein said core is an elastomeric core.
